(19) United States
(12) Patent Application Publication
(10) Pub. No.: US 2004/0198645 A1
(43) Pub. Date: Oct. 7, 2004

(54) Pharmaceutical compositions

(76) Inventors: Michael Ambuhl, Rheinfelden (CH); Barbara Luckel, Lorrach (DE); Jean-Daniel Bonny, Fullinsdorf (CH); Olivier Lambert, Steinbrunn-le-Haut (FR)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/476,832

(22) PCT Filed: May 8, 2003
(86) PCT No.: PCT/EP02/05110

(30) Foreign Application Priority Data
May 9, 2001 (GB) 0111415.6
May 17, 2001 (GB) 0112089.8
Jun. 15, 2001 (GB) 0114700.8

(51) Int. Cl. A61K 38/13; A61K 9/24

(52) U.S. Cl. 514/11; 424/471

(57) ABSTRACT

The present invention provides a solid pharmaceutical composition, e.g. in form of a tablet, powder or capsule, comprising e.g. a cyclosporin.

The present invention relates to novel galenic compositions, in particular novel galenic compositions comprising a poorly water-soluble drug, e.g. a cyclosporin.

Cyclosporins present highly specific difficulties in relation to administration generally and galenic composition in particular, including in particular problems of stability, drug bioavailability, and variability in inter- and intra-patient dose response.

In order to meet these and related difficulties, in GB patent publication no. 2 222 770 and no. 2 257 359, galenic compositions are disclosed comprising a cyclosporin as active ingredient and which take the form of, inter alia, an emulsion, e.g. microemulsion, or emulsion, e.g. microemulsion, pre-concentrate. Microemulsion pre-concentrates have been developed for commercial use under the trademark Neoral® which may be orally administered in the form of drink solutions or soft gelatine capsules.

There remains a need for formulations comprising a poorly water-soluble drug, e.g. cyclosporin, that can be orally administered in solid form, e.g. tablet, powder or capsules, which is stable and exhibit consistent and effective absorption. Conveniently, the tablets or capsules are of a volume that allows convenient administration, e.g. easy swallowing.

The poorly water soluble drug preferably is a lipophilic drug, e.g. a cyclosporin. The term “poorly water soluble”, as used herein, is understood to mean a solubility in water at 20° C. of less than 1, e.g. 0.01, % weight/volume, e.g. a sparingly soluble to very slightly soluble drug as described in Remington: The Science and Practice of Pharmacy, 19^{th} Edition, Ed. A. R. Gennaro, Mack Publishing Company, US, 1995, vol. 1, p 195.

Cyclosporins to which the present invention applies are any of those having pharmaceutical utility, e.g. as immunosuppressive agents, anti-parasitic agents and agents for the reversal of multi-drug resistance, as known and described in the art, in particular Cyclosporin A (also known as Ciclosporin), Cyclosporin G, [O-(2-hydroxyethyl)-(D)Ser]⁸-Ciclosporin, and [3′-dehydroxy-3′-keto-MeBmt]¹-[Val]²-Ciclosporin. Cyciosporin A is preferred.

In one aspect the present invention provides a composition according to the present invention wherein the cyclosporin Is Cyclosporin A.

In accordance with the present invention it has now surprisingly been found that particularly suitable galenic compositions containing a poorly water-soluble drug, e.g. a cyclosporin, having particularly interesting bioavailability characteristics and reduced variability in inter- and intra-subject bioavailability parameters, e.g. in the form of tablets, capsules or powder, are obtainable using a solid polymer and/or a solid surfactant.

The present invention provides in one aspect a solid pharmaceutical composition, e.g. in form of a tablet, a powder or a capsule, comprising

(1) a poorly water soluble drug, e.g. a cyclosporin, and

(2) a polymer which is solid at room temperature.

The polymer is preferably one which can exist in the form of a, e.g. flowable, powder, having a melting point of e.g. above 40° C., preferably having a melting point and/or a glass transition temperature of above about 80° C.

In accordance with the present invention, it has surprisingly been found that suitable cyclosporin-containing compositions and compositions containing other poorly water-soluble drugs may be obtained based on polymers (2) which are solid at room temperature. The polymer is for example a pH dependent or non-pH dependent polymer. The polymer preferably is a hydrophilic polymer. Conveniently one or a mixture of polymers may be used.

Suitable pH-independent polymers include

2.1 polyvinyl pyrrolidone. A preferred example may be PVP K30, having an approx. molecular weight of 50 000 Daltons, or PVP K12, having an approx. molecular weight of 2 500 Daltons, as known and commercially available under the trade name Kollidon® or Plasdone® (Fiedler, “Lexikon der Hilfsstoffe für Pharmazie, Kosmetik und angrenzende Gebiete”, Editio Cantor Verlag Aulendorf, Aulendorf, 4th revised and expanded edition (1996), 1, p.1256);

2.2 cellulose derivatives such as hydroxypropylmethylcellulose, preferably having a molecular weight of from 10 000 to 1 500 000 Daltons, as known and commercially available under the trade names Pharmacoat® or Methocel® (Fiedler, loc. cit. p.790). A preferred example may be as known and commercially available under the name HPMC 3 cP.

Suitable pH-dependent polymers include:

2.3 cellulose derivatives such as hydroxypropylmethylcellulose phthalate, hydroxypropylmethylcellulose acetate succinate or cellulose acetate phthalate. Preferably, hydroxypropylmethylcellulose phthalate may be used as known and commercially available, e.g. from Shin-Etsu, under the name HPMCP HP50, having a viscosity of 190±20 cP, a methoxy content of 20.0-25.0%, hydroxypropyl content of 5.0-10.0%, and a carboxybenzoyl content of 20.0-24.0%, or HPMCP HP55, having a viscosity of 240±20 cP, a methoxy content of 18.0-22.0%, hydroxypropyl content of 4.0-9.0%, and a carboxybenzoyl content of 27.0-35.0% (Fiedler, loc. cit., p.762). Preferably, hydroxypropylmethylcellulose acetate sucdnate (HPMCAS) may be used as known and commercially available, e.g. from Shin-Etsu. Preferably, cellulose acetate phthalate may be used as known and commercially available, e.g. from Eastman Chemical Company, US, under the trade name C-A-P.

2.4 poly(meth)acrylates, preferably having a molecular weight from about 100 000 to about 400 000 Daltons. Preferably, the polymer is a copolymer which is resistant to gastric juice and soluble in intestinal juices, e.g. a copolymer formed from monomers selected from the group consisting of methacrylic acid, methacrylic acid esters, acrylic acid and acrylic acid esters, or e.g. a copolymer formed from butyl methacrylate, (2-dimethyl-aminoethyl)methacrylate, and methyl methacrylate, e.g. as those known and commercially available under the trade mark Eudragit® from Röhm Pharma GmbH. Especially preferred polymers are the 1:1 copolymer formed from monomers selected from the group consisting of methacrylic acid and methacrylic acid lower alkyl esters, such as the 1:1 copolymer formed from methacrylic acid and methyl methacrylate, available under the trade mark Eudragit® L, e.g. Eudragit® L100, having a molecular weight of about 135 000 Daltons, and the 1:1 copolymer of methacrylic acid and acrylic acid ethyl ester as known and commercially available under the trade mark Eudragit® L100-55, having a molecular weight of about 250 000 Daltons, and the 1:2:1 copolymer formed from butyl methacrylate, (2-dimethylaminoethyl)methacrylate, and methyl methacrylate, available under the trade mark Eudragit® E, having a molecular weight of about 150 000 Daltons.

Although any pharmaceutically acceptable components selected from the group of polymers specified above may be used in the composition of the invention, certain components are preferred. These include polyvinyl pyrrolidones, e.g. PVP K12/K30, hydroxypropylmethylcellulose phthalates, e.g. HPMCP HP50/55, or 1:1 copolymers formed from methacrylic acid and methyl methacrylate, e.g. Eudragit® L100 and L 100-55. Conveniently, one or a mixture of these polymers may be used.

pH-Dependent polymers preferably dissolve at a pH of below about 6, e.g. below about 5.

In the pharmaceutical compositions of the present invention, in a further alternative aspect the constitutional ratio of poorly water-soluble drug (e.g. cyclosporin): polymer may be from about (10 to 50): (90 to 50), e.g. 10 : 90, 20: 80, 30: 70, or 50: 50.

The present invention provides in another aspect a solid pharmaceutical composition, e.g. in form of a tablet, a powder or a capsule, comprising

(1) a poorly water soluble drug, e.g. cyclosporin, and

(3) a surfactant which is solid at room temperature.

The present invention provides in a further aspect a solid pharmaceutical composition, e.g. in form of a tablet, a powder or a capsule, consisting of or consisting essentially of

(1) a poorly water-soluble drug, e.g. cyclosporin, and

(3) a surfactant, which is solid at room temperature.

The surfactant (3) is preferably one which can exist in the form of a, e.g. flowable, powder, having a melting point of e.g. above 40° C.

The surfactant (3) is for example nonionic, ionic or amphoteric surfactant Preferably, the surfactants have solubilizing power for the poorly water-soluble drug, e.g. cyclosporins. In one embodiment the invention provides a composition as described above wherein the surfactant is ionic, e.g. surfactants such as listed below under (3.5). In another embodiment the invention provides a composition as described above wherein the surfactant is nonionic, e.g. surfactants such as listed below under (3.1)-(3.4) and (3.6)-(3.12).

Conveniently one or a mixture of the following surfactants may be used:

3.1 polyoxyethylene alkyl ethers; preferably the alkyl ethers are of C₁₂ to C₁₈ alcohols. Preferably the polymer number is from about 2 to about 150, e.g. about 5 to about 150. Preferably the polymers are polyoxyethylene glycol ethers. Preferred examples include polyoxyl 2-, 10- or 20-cetyl ether or polyoxyl 23-lauryl ether, or polyoxyl 20-oleyl ether, or polyoxyl 2-, 10-, 20- or 100-stearyl ether, as known and commercially available e.g. under the trade mark Brij® from Uniqema. An especially preferred product of this class is e.g. Brij® 35 (polyoxyl 23 lauryl ether), Brij® 58, Brij® 78P (polyoxyl 20 stearyl ether), or Brij® 98 (polyoxyl 20 oleyl ether) and polyethoxylated (20) cetyl ether, e.g. Nikkol® BC-20 TX, (H. Fiedler, loc. cit., pp. 259; “Handbook of Pharmaceutical Excipients”, 2nd Edition, Editors A. Wade and P. J. Weller (1994), Joint publication of American Pharmaceutical Association, Washington, USA and The Pharmaceutical Press, London, England, page 367).

Similar products which may also be used are polyoxyethylene-polyoxypropylene-alkyl ethers, e.g. polyoxyethylene-polyoxypropylene-ethers of C₁₂ to C₁₈ alcohols, e.g. polyoxyethylen-20-polyoxypropylene-4-cetylether which is known and commercially available under the trade mark Nikkol PBC® 34, from e.g. Nikko Chemicals Co., Ltd. (Fiedler, loc. cit., vol. 2, pp.1239).

3.2 polyethoxylated fatty acid esters. Preferably the molecular weight is from about 600 to about 18 000 Daltons. Preferably the polymerization number is from about 8 to about 400. Preferably the fatty acid is of 12 to 20 carbon atoms, e.g. stearic acid, e.g. of the type known and commercially available under the trade name Myrj® from Uniqema (Fiedler, loc. cit., vol. 2, pp. 1042). An especially preferred product of this class is Myrj® 52 having a D²⁵ of about 1.1, a melting point of about 40 to 44° C., an HLB value of about 16.9, an acid value of about 0 to 1 and a saponification no. of about 25 to 35, or Myrj® 53, or Myrj® 59 (polyethyleneglycol-100-stearate), e.g. from Uniqema.

3.3 polyethoxylated sorbitan monostearates, e.g. as known and commercially available under the trade name Tween® 61 from Uniqema (Fiedler, loc. cit., vol. 2, pp. 1616).

3.4 polyethoxylated distearates, e.g. as known and commercially available under the trade name Atlas® G 1821 from Uniqema (Fiedler, loc. cit., vol. 2, pp. 206), or Nikko® CDS-6000P from Nikko Chemicals Co., Ltd.

3.5 anionic surfactants, e.g. those based on an alkali metal salt (e.g. of sodium);

3.5.1 sodium alkyl sulfates e.g. sodium C₈-C,₈alkyl sulfates, e.g. sodium C₁₀-C₁₈alkyl sulfates, e.g. sodium lauryl sulfate, which is also known as sodium dodecyl sulfate and which is commercially available, e.g. under the trade name Texapon K12® from Henkel KGaA (Fiedler, loc. cit., vol. 2, pp. 1551);

3.5.2 sodium alkyl sulfonates, e.g. sodium C₈-C₁₈alkyl sulfonates, e.g. sodium C₁₀-C₁₈alkyl sulfonates;

3.5.3 sodium alkyl aryl sulfonates, e.g. sodium C₈-C₁₈alkyl aryl sulfonates, e.g. sodium C₁₀-C₁₈alkyl aryl sulfonates, wherein aryl is e.g. benzyl, phenyl and the like;

3.5.4 sodium alkyl phosphate e.g. sodium C₈-C₁₈alkyl phosphate, e.g. sodium C₁₀-C₁₈alkyl phosphate, e.g. sodium lauryl phosphate, or e.g. potassium cetyl phosphate, available under the trade name of AMPHISOL K from Hoffmann La Roche Ltd.;

3.5.5. sodium stearoyl lactylate (sodium-O-stearyllactate), e.g. as known and commercially available under the name SSL P55 VEG from Danisco; or

3.5.6 sodium (C₄-C₁₂) fatty acid salts e.g. sodium caprinate (Fiedler, loc. cit., vol. 2, pp. 1051).

3.6 polyoxyethylene(POE)polyoxypropylene(POP)-polyoxyethylene(POE) surfactants, e.g. poloxamers, e.g. poloxamer 188, as known and commercially available under the tradename of Pluronic® F 68 from BASF or Synperonic® PE/F 68 from Uniqema, or e.g. poloxamer 407 as known and commercially available under tradename Pluronic® F 127 from BASF or Synperonic PE/F 127 from Uniqema.

3.7 vitamin E based surfactants, e.g. as known and commercially available under the name Vitamin E TPGS (polyethoxylated tocopherol succinate) from e.g. Eastman Kodak.

3.8 sucrose esters, e.g. sucrose stearate or sucrose palmitate.

3.9 monoglyceride based food emulsifiers, e.g. as known and commercially available under the trade name Panodan® AM VEG from Danisco (Fiedler, loc. cit., vol. 2, pp. 1139), or citric acid esters of monoglyceride, e.g. Citrem® LC VEG from Danisco.

3.10 polyethoxylated hydrogenated castor oil, e.g. as known and commercially available under the trade name Cremophor® RH 60 from BASF (Fiedler, loc. cit., vol. 2, pp. 394), which has a saponification value of about 40 to 50, an acid value less than about 1, an iodine value of less than about 1, a water content (Fischer) of about 4.5 to 5.5%, an n_{D}⁶⁰ of about 1.453 to 1.457 and an HLB of about 15 to 17.

3.11 polyethylene glycol (PEG) sterol ethers having, e.g. from 5 to 35 [CH₂—CH₂—O] units, e.g. 20 to 30 units, also in combination with polyoxethylene alkyl ethers. Preferably the polymer is as known and commercially available under the trade name Solulan® C24 (Choleth 24 (and) Ceteth 24) from Amerchol (Fiedler, loc. cit., vol. 2, pp. 1413), or Forlan® C-24 (Choleth 24 (and) Ceteth 24) from R.I.T.A. Corp. (Fiedler, loc. cit., vol. 2, pp. 647)

Similar products which may also be used are those which are known and commercially available under the trade name Nikkol® BPS-30 (polyethoxylated 30 phytosterol) or Nikkol® BPSH-25 (polyethoxylated 25 phytostanol), from e.g. Nikko Chemicals Co., Ltd.

3.12 lecithins, e.g. soy bean phospholipid, e.g. as known and commercially available under the trade name Lipoid® S75 from Lipoid; or egg phospholipid, e.g. as known and commercially available under the trade name Phospholipon® 90 from Nattermann (Fiedler, loc. cit., vol. 2, pp. 1185)

It is to be appreciated that surfactants may be complex mixtures containing side products or unreacted starting products involved in the preparation thereof, e.g. surfactants made by polyoxyethylation may contain another side product, e.g. polyethylene glycol.

In the compositions of the present invention, a surfactant having a hydrophilic-lipophilic balance (HLB) value of 8 to 40, e.g. 8 to 17, is preferred. The surfactant selected preferably has a hydrophilic-lipophilic balance (HLB) of at least 10. The HLB value is preferably the mean HLB value. Preferably, the surfactant is a polyethylene glycol (PEG) sterol ether having from 5 to 35 [CH₂—CH₂—O] units, e.g. Solulan® C24, a polyethoxylated fatty acid ester, e.g. Myrj® 59, a polyoxyethylene alkyl ether, e.g. Brij® 78P, sodium caprinate, or sodium stearoyl lactylate SSL P55.

In a further alternative embodiment, in the pharmaceutical compositions of the present invention consisting of or consisting essentially of (1) a drug and (3) a surfactant, the constitutional ratio of drug (e.g. cyclosporin): surfactant may be e.g. from about 1:0.1 to 20, preferably from about 1:0.1 to 9.

Preferably in the pharmaceutical compositions of the present invention consisting of or consisting essentially of (1) a drug and (3) a surfactant, the surfactant may be selected from the group consisting of surfactants (3.1), (3.2), (3.5) and (3.11). More preferably, the surfactant is a polyethylene glycol (PEG) sterol ether having from 5 to 35 [CH₂—CH₂—O] units, e.g. Solulan® C24, a polyethoxylated fatty acid ester, e.g. Myrj® 59, a polyoxyethylene alkyl ether, e.g. Brij® 78P, sodium caprinate or sodium stearoyl lactate SSL P55. Even more preferably, the surfactant is sodium caprinate or sodium stearoyl lactate SSL P55.

The surfactant may be present in an amount by weight of e.g. 1% up to about 90%, e.g. 10 to 70%, by weight of the composition.

Compositions comprising anionic surfactants, e.g. sodium caprinate or sodium stearoyl lactate SSL P55, preferably are enteric coated. The enteric coating may be applied to tablets and/or to granules, pellets, powders or particles which may be further compressed to tablets.

The term “enteric coating”, as used herein, comprises any pharmaceutically acceptable coating preventing the release of the poorly water-soluble drug in the stomach and sufficiently disintegrating in the intestinal tract, e.g. by contact with juices of a pH of about 5, approximately neutral or alkaline intestine juices, to allow the resorption of the active agent through the walls of the intestinal tract. Preferably, the poorly water-soluble drug, e.g. cyclosporin, is released at a pH of about 5. In vitro tests for determining whether or not a coating is classified as an enteric coating is known in the art.

More specifically, the term “enteric coating”, as used herein, refers to a coating which remains intact for at least 2 hours, in contact with artificial gastric juices such as HCl of pH 1 at 36 to 38° C. and preferably thereafter disintegrates within 30 minutes in artificial intestinal juices such as a KH₂PO₄ buffered solution of pH 6.8.

The enteric coating may be applied as described e.g. in Remington's Pharmaceutical Sciences, 18th Edition, Ed.: Alfonso R. Gennaro, Easton, Pa.: Mack, 1990, Bauer K., Lehmann K., Osterwald H., Überzogene Arzneiformen, 1988, Wissensch. V G, Stuttgart, the contents of which are incorporated herein.

Preferably, the release of the poorly water-soluble drug is not prolonged by the enteric coating.

In another embodiment, the compositions of the invention, e.g. in form of a tablet, a powder or a capsule, comprise

(1) a poorly water soluble drug, e.g. a cyclosporin,

(2) a polymer which is solid at room temperature, and

(3) a surfactant, e.g. a nonionic or ionic or amphoteric surfactant.

The surfactant may be selected from the group (3.1) to (3.12) mentioned above. Preferably a non-ionic surfactant may be used. More preferably, the surfactant may be selected from the group consisting of surfactants (3.1), (3.2), and (3.11). Even more preferably, the surfactant is a polyethylene glycol (PEG) sterol ether having from 5 to 35 [CH₂—CH₂—O] units, e.g. Solulan® C24, a polyethoxylated fatty acid ester, e.g. Myrj® 59, and a polyoxyethylene alkyl ether, e.g. Brij® 78P.

In a further aspect the present invention provides the compositions of the invention, e.g. in form of a tablet, a powder or a capsule, comprising

(1) a poorly water soluble drug, e.g. a cyclosporin,

(2) a polymer which is solid at room temperature, and

(3) a surfactant, which e.g. is solid at room temperature, e.g. a surfactant which can exist in the form of a, e.g. flowable, powder and having a melting point of e.g. above 40° C.

In the pharmaceutical composition of the present invention comprising (1) a poorly water soluble drug, e.g. a cyclosporin, (2) a polymer which is solid at room temperature, and (3) a surfactant, the amount of the surfactant may be up to about 50%, e.g. up to about 40%, e.g. up to about 20% by weight, e.g. 1 to 15% by weight, preferably from about 2 to 10, In particular about 3 to 7% by weight based on the total weight of the composition comprising the poorly water-soluble drug, e.g. cydosporin, the polymer and the surfactant Preferably, the ratio of surfactant: drug (e.g. cyclosporin) is 1:0.5 to 50, e.g. 1:1 to 40, e.g. 1:2 to 20. Preferably these three components comprise at least 95, or 95% of the composition.

A preferred embodiment comprises cyclosporin compositions comprising a polymer (2) which is solid at room temperature, and a surfactant (3) which is solid at room temperature.

In a further aspect, the invention provides a pharmaceutical composition e.g. in form of a tablet, a powder or a capsule comprising

(1) a poorly water soluble drug, e.g. a cyclosporin,

(2) a polymer,

(3) optionally a surfactant and

(4) a carrier.

In another aspect, the invention provides a pharmaceutical composition e.g. in form of a tablet, a powder or a capsule consisting of or consisting essentially of

(1) a poorly water soluble drug, e.g. a cyclosporin,

(3) a surfactant and

(4) a carrier.

Preferably as a carrier is present e.g.:

4.1 a water-soluble or water-insoluble saccharide such as lactose or mannitol;

4.2 microcrystalline cellulose, e.g. as known and commercially available under the trade name Avicel® from FMC Corporation; or

4.3 colloidal silicon dioxide, e.g. as known and commercially available under the trade name Aerosil®;

4.4 anhydrous calcium phosphate, e.g. as known and commercially available under the trade name Fujicalin®, or anhydrous dicalcium phosphate, e.g. as known and commercially available under the trade name A-TAB® from Rhodia.

A mixture of carriers may be present.

Any carrier, if present, is generally present in an amount of up to about 50%, e.g. 0.5 to 50%, e.g. 10 to 40%, e.g. 15 to 40% by weight, preferably from about 20 to about 30% by weight based on the total weight of the composition comprising the drug, e.g. cyclosporin, the polymer and/or surfactant and the carrier.

The surfactant is preferably present in an amount of 20 to 50% by weight of the composition, for example about 30% by weight of the composition comprising the drug, e.g. cyclosporin, polymer and/or the surfactant and the carrier.

In another aspect, the invention provides a pharmaceutical composition e.g. in form of a tablet, a powder or a capsule comprising

(1) a poorly water soluble drug, e.g. a cyclosporin, e.g. cyclosporin A,

(2) a polymer,

(3) optionally a surfactant,

(4) optionally a carrier, and

(5) optionally a disintegrant.

In yet another aspect, the invention provides a pharmaceutical composition e.g. in form of a tablet, a powder or a capsule consisting of or consisting essentially of

(i) a poorly water soluble drug (1), e.g. a cyclosporin, e.g. cyclosporin A,

(ii) a surfactant (3),

(iii) a carrier (4), and/or a disintegrant (5).

Suitable disintegrants include e.g.

5.1 natural starches, such as

5.1.1 maize starch, potato starch, and the like,

5.1.2 directly compressible starches, e.g. Sta-rx® 1500, modified starches, e.g. carboxymethyl starches and sodium starch glycolate, available as Primojel®, Explotab®, Explosol®, and

5.1.3 starch derivatives such as amylose;

5.2 crosslinked polyvinylpyrrolidones, e.g. crospovidones, e.g. Polyplasdone® XL and Kollidon® CL;

5.3 alginic acid or sodium alginate;

5.4 methacrylic acid-divinylbenzene copolymer salts, e.g. Amberlite® IRP-88; and

5.5 cross-linked sodium carboxymethylcellulose, available as e.g. Ac-di-sol®, Primellose®, Pharmacel® XL, Explocel®, and Nymcel® ZSX, or

5.6 a mixture of thereof.

The disintegrant or disintegrants may be present in an amount of 1 to 50%, e.g. 5 to 40% by weight based on the total weight of the composition.

In a further aspect, the invention provides a pharmaceutical composition e.g. in form of a tablet, a powder or a capsule comprising

(1) a poorly water soluble drug, e.g. a cyclosporin, e.g. cyclosporin A,

(2) a polymer,

(3) optionally a surfactant,

(4) optionally a carrier,

(5) optionally a disintegrant, and

(6) optionally a lubricant, e.g. magnesium stearate.

In yet another aspect, the invention provides a pharmaceutical composition e.g. in form of a tablet, a powder or a capsule consisting of or consisting essentially of

(i) a poorly water soluble drug (1), e.g. a cyclosporin, e.g. cyclosporin A,

(ii) a surfactant (3),

(iii) a carrier (4), a disintegrant (5) and/or a lubricant (6), e.g. magnesium stearate.

Lubricants may be present in a total amount of up to about 5% by weight, e.g. 2%, e.g. 1% by weight based on the total weight of the composition.

The pharmaceutical composition may also include further additives or ingredients, for example antioxidants, such as ascorbyl palmitate, butyl hydroxy anisole (BHA), butyl hydroxy toluene (BHT) and tocopherols, and/or preserving agents. In a further alternative aspect these additives or ingredients may comprise about 0.05 to 1% by weight of the total weight of the composition. The pharmaceutical composition may also include sweetening or flavoring agents in an amount of from e.g. 0.1 to e.g up to about 2.5 or 5% by weight based on the total weight of the composition.

Details of excipients of the invention are described in e.g. Fiedler, H. P., loc cit; “Handbook of Pharmaceutical Excipients”, loc cit: or may be obtained from the relevant manufacturers, the contents of which are hereby incorporated by reference.

Preferably the compositions of the present invention do not contain any organic hydrophilic component. Under “organic hydrophilic component” is to be understood any hydrophilic component or any hydrophilic co-component as described in the above mentioned British patent application no. 2 222 770. Such hydrophilic components excluded may comprise no added hydrophilic component such as water soluble components and/or ethanol, propylene glycol or water. Naturally it will be appreciated that small amounts of organic hydrophilic components e.g. which have no significant effect, may be tolerated, e.g. as a result of impurities such as less than 3% by weight of the composition.

Preferably the compositions of the present invention do not contain any lipophilic component. Under “lipophilic component” is to be understood any lipophilic component as described in the above mentioned British patent application no. 2 222 770. Such lipophilic components excluded comprise no added lipophilic component such as glyceryl fatty acid ester. Naturally it will be appreciated that small amounts of lipophilic components e.g. which have no significant effect, may be tolerated, e.g. as a result of impurities such as less than 3% by weight of the composition.

Accordingly, in one aspect the present invention provides a composition as described above which is free, e.g. substantially free, from an organic hydrophilic component and/or a lipophilic component. In one group of compositions of the present invention there is no glyceryl fatty acid present.

The drug, e.g. cyclosporin, may be present in an amount by weight of up to about 50% by weight of the composition. The drug is preferably present in an amount of e.g. 1 to 50%, e.g. 15 to 40% by weight of the composition, for example about 20% by weight of the composition comprising the drug, e.g. cydosporin, the polymer and/or the surfactant. Yet, the tablets or capsules are of a volume that allows convenient administration, e.g. easy swallowing.

In one aspect, upon dilution with an aqueous medium the compositions of the present invention may form, e.g. to an substantial amount, e.g. to the extent of 60% or more, e.g. 85% or more, e.g. more than 90, 95 or 99%, fine partides of, e.g. substantially amorphous, poorly water-soluble drug, e.g. cyclosporin. By “substantially amorphous” is meant more than 90%, e.g. more than 95%, preferably about or more than 99% in amorphous form.

Preferably, upon dilution with an aqueous medium, for example water, for example on dilution of 1:1 to 1:300, e.g. 1:5 to 1:100, e.g. 1:10 to 1:100, or in the gastricjuices after oral application, the compositions of the present invention, comprising (1) a poorly water soluble drug, e.g. a cyclosporin, (2) a polymer and/or (3) a surfactant, spontaneously substantially form fine particles, e.g. solid particles of substantially amorphous poorly water-soluble drug, e.g. cyclosporin, e.g. of a range of from 50 nm to 20 000 nm, e.g. from 50 nm to 10 000 nm, e.g. from 50 nm to 2000 nm, e.g. as measured by conventional methods, e.g. light diffraction techniques, e.g. based on a Mastersizer. Conveniently, there is a narrow size distribution.

In another aspect, upon dilution with an aqueous medium the compositions of the present invention comprising (1) a poorly water soluble drug, e.g. cyclosporin, (3) a surfactant which is solid at room temperature, may form a system which is a mixture of substantially solubilized drug, e.g. about 10 to 100%, preferably about 10 to 80%, e.g. 30 to 40%, more preferably 40 to 70% of the total drug and particulate drug, e.g. about 0 to 90%, preferably about 20 to 90%, e.g. 60 to 70%, more preferably 30 to 60% of the total drug. The constitutional ratio of drug : surfactant may be preferably 1:0.1, or 1:0.25, or 1:0.5, or 1:1, or 1:2, or 1:4, or 1:9. Preferably, the drug is cyclosporin, e.g. cydosporin A.

In yet a further aspect the present invention provides compositions which upon dilution with an aqueous medium form a system wherein the poorly water-soluble drug, e.g. cydosporin, e.g. Cydosporin A, substantially is solubilized, e.g. is solubilized to an extent of about 90% of total drug or more, e.g. more than about 95%. It has been found that surprisingly low drug (e.g. cydosporin): nonionic surfactant ratios of e.g. about 1:5.3 to 6.6, may be used to completely solubilize the drug, e.g. cyclosporin, when one of the nonionic surfactants as specified above, e.g. Choleth 24 (and) Ceteth 24, e.g. Solulan® C24 or Forlan® C-24; or polyethoxylated (30) phytosterol, e.g. Nikkol® BPS30; or polyethoxylated (25) phytostanol, e.g. Nikkol® BPSH-25; or polyethoxylated (20) stearyl ether, e.g. Brij® 78P; or polyethoyxlated (20) cetyl ether, e.g. Nikkol® BC-20 TX, is used. Particularly suitable are polyethoxylated (30) phytosterol, e.g. Nikkol® BPS-30; or polyethoxylated (25) phytostanol, e.g. Nikkol® BPSH-25; or polyethoxylated (20) stearyl ether, e.g. Brij® 78P.

The amount of poorly water-soluble drug, e.g. cyclosporin, which can be solubilized may be analyzed by centrifugation followed by HPLC for the distribution of drug, e.g. cyclosporin, between the solubilized and particulate phase.

The state of the particles may be analyzed by X-ray and the particle size distribution may be analyzed e.g. by laser light scattering or electron microscopy.

The compositions of this invention may produce on contact with water stable e.g. particulate systems, e.g. for up to one day or longer, e.g. one day. Preferably the systems remain stable for more than 5 hours.

In one aspect the present invention provides a composition, comprising (1) a poorly water-soluble drug, e.g. cyclosporin, (2) a polymer and/or (3) a surfactant, which is in form of a solid dispersion.

In a further alternative aspect the present invention provides a composition according to the present invention comprising (2) a polymer wherein the poorly water-soluble drug, e.g. cyclosporin, is encapsulated in a polymeric matrix, e.g. in form of microparticles.

The compositions of the invention may be prepared by working up active agent with the excipients. The following processes A to H are contemplated.

A. In one aspect the compositions of the present Invention in form of a solid dispersion comprising (1) a poorly water-soluble drug, e.g. cyclosporin, (2) a polymer and/or (3) a surfactant may be obtained by

(i) dissolving, suspending or dispersing the drug, e.g. cyclosporin, and polymer, if present, in a solvent or solvent mixture,

(ii) adding the surfactant, if present, to the drug/solvent or drug/polymer/solvent mixture,

(iii) evaporating the solvent and co-precipitating the drug, e.g. cyclosporin, with the polymer and/or the surfactant,

(iv) drying the resulting residue, e.g. under reduced pressure, milling and sieving the particles.

The solvent of (i) may be a single solvent or a mixture of solvents. Suitable solvents for use according to the present invention may be organic solvents such as an alcohol, e.g. methanol, ethanol, or isopropanol; an ester, e.g. ethylacetate; an ether, e.g. diethylether, a ketone, e.g. acetone; or a halogenated hydrocarbon, e.g. dichloromethane. Preferably a solvent mixture of ethano/acetone having a weight ratio of ethanol : acetone of between about 1:10 to about 10:1, e.g. 1:5 to 5:1 may be used.

B. In another aspect the compositions of the present invention in form of a solid dispersion comprising (1) a poorly water-soluble drug, e.g. cyclosporin, (2) a polymer and/or (3) a surfactant may be obtained by

(i) dissolving, suspending or dispersing the drug, e.g. cyclosporin, and surfactant, if present, in a solvent or solvent mixture and optionally adding small amounts of water, if necessary,

(ii) adding the polymer, if present, to the drug/solvent or drug/surfactant/solvent mixture,

(iii) evaporating the solvent and precipitating the drug, e.g. cyclosporin, with the surfactant and/or the polymer,

(iv) drying the resulting residue, e.g. under reduced pressure, milling and sieving the particles.

The solvent of (i) may be a single solvent or a mixture of solvents. Suitable solvents for use according to the present invention may be organic solvents such as an alcohol, e.g. methanol, ethanol, or isopropanol; an ester, e.g. ethylacetate; an ether, e.g. diethylether, a ketone, e.g. acetone; or a halogenated hydrocarbon, e.g. dichloromethane. Preferably a solvent mixture of ethanol/acetone having a weight ratio of ethanol : acetone of between about 1:10 to about 10:1, e.g. 1:5 to 5:1 may be used.

C. Alternatively, the solid dispersions of the invention, comprising (1) a poorly water-soluble drug, e.g. cyclosporin, (2) a polymer and/or (3) a surfactant, may be prepared by spray-drying techniques. A solution or dispersion as formed above is dispersed through a nozzle at an inlet temperature of about 50 to about 130° C. into a chamber. The solvent is evaporated through the nozzle, and finely dispersed particles are collected.

D. In a further alternative embodiment of the present invention the solid dispersion, comprising (1) a poorly water-soluble drug, e.g. cyclosporin, (2) a polymer and/or (3) a surfactant, may be prepared by spray-drying the solution or dispersion as formed above onto (4) a carrier in the fluid bed.

The particles typically have a mean particle size of less than about 2 mm, e.g. 1 mm, e.g. 0.5 mm, as measured e.g. by light microscopy.

E. The compositions of the present invention wherein the poorly water-soluble drug, e.g. cyclosporin, is encapsulated in a polymeric matrix, e.g. in form of microparticdes, may be prepared e.g. according to a process comprising the following steps:

(i) preparation of an internal organic phase comprising

(ia) dissolving the polymer in an organic solvent or solvent mixture. The solvent may be a single solvent or a mixture of solvents. Suitable solvents for use according to the present invention may be organic solvents such as a ketone, e.g. acetone; or a halogenated hydrocarbon, e.g. methylene chloride. Preferably a solvent mixture of methylene chloride/acetone having a weight ratio of methylene chloride : acetone of between about 1:10 to about 10:1, e.g. 1:5 to 5:1, preferably 1:1, may be used,

(ib) adding the poorly water-soluble drug, e.g. cyclosporin, to the polymer solution, and optionally

(ic) adding a surfactant to the solution obtained by step (ib),

(ii) preparation of an external aqueous phase comprising

(iia) preparing a buffer, e.g. acetate buffer,

(iib) dissolving gelatin or polyvinylalcohol (PVA) in water, and

(iic) mixing the solution obtained by step (iib) with the solution obtained by step (iia) to obtain e.g. a 0.5% gelatin solution In the buffer,

(iii) mixing the internal organic phase, e.g. brought at 20 ml/min with a gear pump, with the external aqueous phase, e.g. brought at 400 ml/min with a gear pump, e.g. in a ratio of internal phase to external phase of about 1:10 to about 1:40, preferably about 1:20, with a device creating high shear forces, e.g. with a static mixer, to form e.g. an oil/water emulsion, and

(iv) hardening the microparticles by solvent evaporation, washing for excipients removal and collecting the microparticles.

The microparticles typically have a mean particle size of less than about 350 microns, e.g. about 1 to about 180 microns, as measured e.g. by scanning electron microscopy.

In order to e.g. increase flowability of the final microparticle powder, the obtained microparticles may be further worked up by adding an aqueous solution of a carrier, e.g. lactose, and lyophilization or spray drying of the resulting suspension to obtain a, e.g. flowable, powder.

F. In one embodiment the compositions of the invention, in form of solid dispersions, comprising a surfactant are obtained by

(i) preparation of an organic preconcentrate comprising dissolving the surfactant in an organic solvent or a mixture of solvents, e.g. ethanol, adding the poorly water-soluble drug, e.g. cyclosporin, and stirring until dissolved,

(ii) diluting or delivering the organic preconcentrate obtained in step (i) to a mixer, e.g. a magnetic stirrer or a static mixer, together with an aqueous solution, optionally comprising a carrier, e.g. lactose, and

(iii) spray-drying the mixture or, if no carrier is present in step (ii), spray-drying the diluted preconcentrate obtained in step (ii) onto a carrier, e.g. lactose, e.g. in the fluid bed.

G. In yet a further embodiment of the present invention the compositions of the invention, in form of solid dispersions, comprising a surfactant (3) are prepared by

(i) dissolving the surfactant, e.g. ionic surfactant, the cycloysporin and optionally a carrier e.g. lactose in water, and

(ii) spray-drying the aqueous solution

H. In yet a further alternative embodiment of the present invention the compositions of the invention, in form of solid dispersions, comprising a surfactant (3) are prepared by

(i) dissolving the poorly water-soluble drug, e.g. cyclosporin, in an organic solvent, e.g. propylene glycol, to obtain e.g. a 40% solution of poorly water-soluble drug, e.g. cyclosporin, in propylene glycol,

(ii) mixing the solution obtained in step (i) with a molten surfactant,

(iii) optionally mixing or granulating the mixture obtained in step (ii) with a carrier, e.g. lactose; or microcrystalline cellulose, or colloidal silicon dioxide; or anhydrous calcium phosphate, and

(iv) cooling the mixture obtained in step (ii) or (iii) to obtain a solid composition.

The solid dispersions obtained by processes F to H preferably do not contain any polymer (2).

Other excipients may be added at any stage, preferably however after the powder is formed.

The resulting mixtures of any of the processes F to H described above may be dried, milled and sieved to obtain a fine, e.g. flowable, powder.

The compositions of the invention in powder form, e.g. particles, e.g. solid dispersion particles or microparticles, may be compressed to tablets.

The particles, e.g. solid dispersion particles or microparticles, may be combined with one or more flow enhancers, e.g. colloidal silicon dioxide, and/or one or more solid surfactants as specified above, e.g. sodium lauryl sulfate, e.g. in a total amount of enhancers and/or surfactants of up to about 70% by weight, e.g. 20 to 60% by weight, in particular 40 to 50% by weight based on the total weight of the composition.

If present in the compositions, the filler or a mixture of fillers, the disintegrants or a mixture of disintegrants, the lubricants or a mixture of lubricants, the flow enhancers or a mixture of flow enhancers, the additional surfactant or surfactants may be added to the drug/polymer/solvent mixture, the drug/surfactant'solvent mixture, the drug/polymer/surfactant/solvent mixture or, preferably, to the outer tabletting phase.

In one aspect of the invention the outer tabletting phase may comprise one or more solid surfactants as specified above, e.g. sodium lauryl sulfate, instead or in addition to adding a surfactant to the drug/polymer/solvent mixture in the preparation process of the solid dispersion particles or microparticles, comprising (1) a poorly water-soluble drug, e.g. cyclosporin, (2) a polymer and optionally (3) a surfactant, as hereinabove described.

The outer tabletting phase may comprise e.g. spray-dried lactose/microcrystalline cellulose mixtures, dicalcium phosphate anhydrous or a mixture of α-lactose monohydrate and microcrystalline cellulose, e.g. Microcelac® 100, e.g. to achieve tablet compositions with a suitable average hardness and a short disintegration time.

Microcelac® 100 is a spray-dried compound consisting of 75% α-lactose monohydrate and 25% microcrystalline cellulose produced by Meggle.

Accordingly, in one embodiment, the present invention provides tablet compositions with an average hardness of e.g. from 60 N to 200 N, preferably 80 N to 110 N, and/or a disintegration time of e.g. below about 10 min, preferably below 1 min, wherein the outer tabletting phase comprises e.g. lactose/microcrystalline cellulose mixtures, dicalcium phosphate anhydrous or α-lactose monohydrate/microcrystalline cellulose mixtures.

Preferably, the compositions comprise a-lactose monohydrate/microcrystalline cellulose mixtures, e.g. Microcelac® 100, in an amount of e.g. about 10 to 80%, e.g. about 10 to 60% by weight based on the total weight of the composition or dicalcium phosphate anhydrous in an amount of e.g. about 10 to 80%, e.g. about 10 to 60% by weight based on the total weight of the composition.

Preferably, compositions comprising HPMCP comprise α-lactose monohydrate/mmicrocrystalline cellulose mixtures, e.g. Microcelac®. Preferably, compositons comprising PVP comprise dicalcium phosphate anhydrous.

Applicants have found that surprisingly high drug loadings may be obtained in accordance with the present invention, e.g. drug loadings up to 70%, e.g. from about 20 to about 60%, in particular about 30 to 50% by weight based on the total weight of the particles, e.g. solid dispersion particles or microparticles, or e.g. drug loadings of up to 40%, e.g. about 20% by weight based on total weight of the final composition.

The compositions, e.g. those in the examples hereinafter, show good stability characteristics as indicated by standard stability trials, e.g. no poorly water-soluble drug, e.g. cyclosporin, crystallization (as determined by differential scanning calorimetry) or degradation, having e.g. a shelf life stability of up to one, two or three years, and even longer. The compositions of this invention may produce stable particulate systems upon dilution with aqueous media, e.g. for up to one day or longer, e.g. one day.

The pharmaceutical compositions of the invention exhibit especially advantageous properties when administered orally; for example in terms of consistency and high level of bioavailability obtained in standard bioavailability trials. These trials are performed in animals e.g. rats or dogs or healthy volunteers using HPLC or a specific or nonspecific monoclonal kit to determine the level of the drug substance, e.g. cyclosporin in the blood. For example, the compositions of Examples 1 to 15 administered p.o. to dogs may give surprisingly high Cₘₐₓ and AUC(0-24h) values as detected by a radioimmunoassay (RIA) method using a specific monoclonal antibody and within e.g. 60 to 120%, preferably 90 to 120%, of that of Neoral®.

In one aspect the present invention provides a method of orally administering a pharmaceutical composition, said method comprising orally administering to a patient in need of poorly water-soluble drug, e.g. cyclosporin, therapy a composition according to the present invention.

Pharmacokinetic parameters, for example absorption and blood levels, also become surprisingly more predictable and problems in administration with erratic absorption may be eliminated or reduced. Additionally the pharmaceutical compositions are effective with biosurfactants or tenside materials, for example bile salts, being present in the gastro-intestinal tract. That is, the pharmaceutical compositions of the present invention are fully dispersible in aqueous systems comprising such natural tensides and thus capable of providing particulate systems in situ which are stable. The function of the pharmaceutical compositions upon oral administration remain substantially independent of and/or unimpaired by the relative presence or absence of bile salts at any particular time or for any given individual.

The pharmaceutical compositions of the invention release the poorly water-soluble drug, e.g. cyclosporin, to the extent of e.g. about above 80% over a 60 minute period, e.g. about 75% in a 15 minute period, as measured by standard in vitro dissolution studies, e.g. at pH 6.8 or 1 using the paddle method.

The compositions of this invention show reduced variability in inter- and intra-patient dose response.

In one aspect the present invention provides a method of reducing the variability of bioavailability levels of a poorly water-soluble drug, e.g. cyclosporin, for patients during poorly water-soluble drug, e.g. cyclosporin, therapy, said method comprising orally administering an oral pharmaceutical composition according to the present invention.

The utility of all the pharmaceutical compositions of the present invention may be observed in standard clinical tests in, for example, known indications of drug dosages giving equivalent blood levels of drug; for example using dosages in the range of 2.5 mg to 1000 mg of drug per day for a 75 kilogram mammal, e.g. adult and in standard animal models. The increased bioavailability of the drug provided by the compositions may be observed in standard animal tests and in clinical trials, e.g. as described above.

The optimal dosage of drug to be administered to a particular patient may be considered carefully as individual response to and metabolism of the drug, e.g. cyclosporin, may vary, e.g. by monitoring the blood serum levels of the drug by radioimmunoassay (RIA), enzyme linked immunosorbent assay (ELISA), or other appropriate conventional means. Poorly water-soluble drug, e.g. cyclosporin, dosages may be 25 to 1000 mg per day (preferably 50 mg to 500 mg).

The pharmaceutical composition, e.g. in form of a tablet or a powder suitable for tablet formation, will suitably contain between 10 and 100 mg of the drug, for example 10, 15, 20, 25, 50, or 100 mg. Such unit dosage forms are suitable for administration 1 to 5 times daily depending upon the particular purpose of therapy, the phase of therapy and the like.

The pharmaceutical compositions of the invention are useful for the same indications as the poorly water soluble drugs. The pharmaceutical compositions comprising a cyclosporin are particularly useful for

a) treatment and/or prevention of organ, cell or tissue transplant rejection, for example for the treatment of the recipients of heart, lung, combined heart-lung, liver, kidney, pancreatic, skin or corneal transplants. The pharmaceutical compositions are also indicated for the prevention of graft-versus-host disease, such as sometimes occurs following bone marrow transplantation;

b) treatment and/or prevention of autoimmune disease and of inflammatory conditions, in particular inflammatory conditions with an aetiology including an autoimmune component such as arthritis (for example rheumatoid arthritis, arthritis chronic progrediente and arthritis deformans) and rheumatic diseases; and

c) treatment and/or prevention of psoriasis.

Pharmaceutical compositions of the invention, e.g. comprising cyclosporin, may be used alone or together with other immunosuppressants, immunomodulatory or anti-inflammatory drugs. For example, they may be used in combination with everolimus, sirolimus, tacrolimus, pimecrolimus, mycophenolic acid, mycophenolate sodium, mycophenolate mofetil, an accelerating lymphocyte homing agent, e.g. FTY720, corticosteroids, or the like.

Therefore in a further aspect the present invention provides

i. a pharmaceutical composition, e.g. comprising cyclosporin, as defined above for use in the treatment and/or prevention of organ, cell or tissue transplant rejection, prevention of graft-versus-host disease, treatment and/or prevention of autoimmune disease and of inflammatory conditions, and treatment and/or prevention of psoriasis;

ii. a method of treating and/or preventing organ, cell or tissue transplant rejection, preventing graft-versus-host disease, treating and/or preventing autoimmune disease and inflammatory conditions, and treating and/or preventing psoriasis, comprising administering a composition of the present invention, e.g. comprising cyclosporin, to a patient in the need thereof;

iii. the use of a composition of the present invention, e.g. comprising cyclosporin, in the preparation of a medicament for the treatment and/or prevention of organ, cell or tissue transplant rejection, prevention of graft-versus-host disease, treatment and/or prevention of autoimmune disease and of inflammatory conditions, and treatment and/or prevention of psoriasis; or

iv. a method as defined above comprising co-administering a composition of the present invention, e.g. comprising cyclosporin, and a second drug substance, said second drug substance being e.g. an immunosuppressant, an immunomodulatory or an anti-inflammatory drug.

Following is a description by way of example only of compositions of this invention. Unless otherwise indicated, components are shown in % by weight based on each composition. The examples illustrate compositions useful for example in the prevention of transplant rejection or for the treatment of autoimmune disease, on administration of from 1 to 5 unit dosages/day at a dose of 2 to 5 mg/kg per day. The examples are described with particular reference to Cyclosporin A but equivalent compositions may be obtained employing any cyclosporin or other poorly water-soluble drug.

### EXAMPLE 1 to 7

Preparation of Solid Dispersion Compositions

Compositions of examples I to 7 in amount as indicated in Table 1 are made up by dissolving Cyclosporin A in an ethanol/acetone mixture, adding the polymer, surfactant, if present, and carrier medium, if present, of Table 1, mixing until homogenously dispersed, evaporation of the solvents, and drying, milling and sieving the resulting residue.

TABLE 1

| COMPONENT | Ex 1 | Ex 2 | Ex 3 | Ex 4 | Ex 5 | Ex 6 | Ex 7 |
|---|---|---|---|---|---|---|---|
| Cyclosporin A | 21% | 30% | 30% | 40% | 20% | 25% | 30% |
| PVP K30 | — | 67% | — | — | — | 72% | — |
| HPMCP HP50 | — | — | 67% | 55% | 75% | — | 63% |
| Eudragit ® L100-55 | 50% | — | — | — | — | — | — |
| Solulan ® | — | 3% | | — | — | 3% | — |
| Myrj ® 59 | — | — | 3% | 5% | 5% | — | — |
| Brij ® 78P | — | — | — | — | — | — | 7% |
| Lactose | 25% | — | — | — | — | — | — |
| Crospovidone | 4% | — | — | — | — | — | — |

### EXAMPLE 8 and 9

Preparation of Microparticule Compositions

Compositions of example 8 and 9 in amounts as indicated in Table 2 are made up by dissolving HPMCP HP50 in methylene chloride/acetone, adding Cyclosporin A and Brij® 78P or Myrj®59, respectively; delivering the polymer system to a mixer together with a buffered gelatin solution; evaporation of the solvent, washing for excipients removal and collecting the microparticles.

TABLE 2

| COMPONENT | Ex 8 | Ex 9 |
|---|---|---|
| Cyclosporin A | 30% | 40% |
| HPMCP HP50 | 63% | 55% |
| Brij ® 78P | 7% | — |
| Myrj ® 59 | — | 5% |

Other examples may be made by replacing Eudragit® L100-55 or HPMCP HP50 by any of the polymers specified above or by replacing Brij® 78P by any of the surfactants specified above.

### EXAMPLE 10

Compositions of example 10 in amounts as indicated in Table 3 are made up by dissolving the surfactant and the cyclosporin and suspending the carrier in ethanol, stirring to obtain a homogenous suspension, and evaporation of the solvent under reduced pressure.

The resulting powder is milled and sieved. After dilution with water at a ratio of 1+100 at 37° C. the distribution of Cyclosporin A between solubilized and particulate phase is analyzed by centrifugation followed by HPLC. The results show a mixture of solubilized (35%) and particulate (65%) cyclosporin A. Particle sizes of up to about 12.5 microns are measured by a light microscope.

TABLE 3
quantity given in wt-%

| COMPONENTS | Ex. 10 | Ex. 11 | Ex. 12 |
|---|---|---|---|
| Cyclosporin A | 25% | 30% | 25% |
| Brij ® 78P | 50% | — | — |
| Sodium stearyl lactylate P55 | — | 30% | — |
| Sodium caprinate | — | — | 37% |
| Lactose | 25% | 40% | 38% |

### EXAMPLE 11

Compositions of example 11 in amounts as indicated in Table 3 are made up by dissolving the surfactant in ethanol, adding the cyclosporin, stirring to obtain a solution, delivering the organic preconcentrate to a mixer together with an aqueous solution of lactose, and spray-drying the mixture to obtain a fine powder.

The resulting powder is diluted with water at a ratio of 1+100 at 37° C. and the distribution of Cyclosporin A between solubilized and particulate phase is analyzed by centrifugation followed by HPLC. The results show a mixture of solubilized (29%) and particulate 71% cyclosporin A. Particle sizes of up to about 2.5 microns are measured by a light microscope.

### EXAMPLE 12

Compositions example 12 in amounts as indicated in Table 3 are made up by dissolving the surfactant, the cyclosporin, and the carrier in water, and spray-drying the aqueous solution to obtain a fine powder.

The resulting powder is diluted with water at a weight ratio of 1+7 at 37° C. and the distribution of Cyclosporin A between solubilized and particulate phase is analyzed by centrifugation followed by HPLC. The results show a mixture of solubilized (72%) and particulate (28%) cyclosporin A.

Other examples may be made by replacing Brij® 78P, or sodium stearoyl lactylate P55, or sodium caprinate by any of the surfactants specified above.

Other examples may be made by replacing lactose by any of the carriers specified above.

### EXAMPLE 13 and 14

Preparation of Tablets Based on Solid Dispersion Particles

Compositions of examples 13 and 14 in amount as indicated in Table 4 are made up by dissolving Cyclosporin A in an ethano/acetone mixture, adding the polymer, surfactant, if present, and carrier medium, if present, of Table 4, mixing until homogenously dispersed, evaporation of the solvents, and drying, milling and sieving the resulting residue. The resulting particles are mixed with the additional excipients and directly compressed to flat tablets.

The tablets have a hardness (compression force), a disintegration time and dissolution rates as indicated in Table 5.

TABLE 4

| COMPONENT | Ex 13 | Ex 14 | Ex. 15 |
|---|---|---|---|
| Cyclosporin A | 16.7% | 14.3% | 20% |
| PVP K30 | — | 41.2% | — |
| HPMCP HP50 | 22.9% | — | 27.5% |
| Solulan ® | — | 1.7% | — |
| Myrj ® 59 | 2.1% | — | 2.5% |
| Crospovidone | 20% | 30% | 20% |
| Microcelac 100 | 37.5% | — | 29.2% |
| dicalcium phosphate anhydrous | — | 12% | — |
| magnesium stearate | 0.5% | 0.5% | 0.5% |
| Aerosil 200 | 0.3% | 0.3% | 0.3% |

TABLE 5

| | Ex 13 | Ex 14 | Ex. 15 |
|---|---|---|---|
| average hardness in N | 91 | 94 | 95 |
| disintegration time in minutes | <1 | <8 | <1 |
| tablet diameter in mm | 10 | 11 | 9 |
| tablet weight in mg | 300 | 350 | 250 |
| dissolution rate after 15 min | 80% | 90% | 89% |
| dissolution rate after 60 min | 92% | 94% | 90% |

### EXAMPLE 15

Preparation of Tablets Based on Microparticules

Compositions of example 15 in amounts as indicated in Table 4 are made up by dissolving HPMCP HP50 in methylene chloride/acetone, adding Cyclosporin A and Myrj®59; delivering the polymer system to a mixer together with a buffered gelatin solution; evaporation of the solvent, washing for excipients removal and collecting the microparticles. The resulting particles are mixed with the additional excipients and directly compressed to flat tablets. The tablets have a hardness (compression force), a disintegration time and dissolution rates as indicated in Table 5.

### EXAMPLE 16

Single oral doses of 50 mg cyclosporin A per animal of composition of example 1, 8, 10, 11 and 12 filled in a hard gelatin capsule size 1, corresponding to about 5 mg/kg were given to fasted dogs (n=8) using a two-block latin square design with a one-week interval between administrations. The nominal doses of cyclosporin A in mg/kg body weight are listed in Table 6.

Blood (about 1 ml each) was collected from the cephalic or jugular vein at 0 min (=pre-dose), and 10 min, 30 min, 45 min, 1 hour, 1.5 hours, 2 hours, 3 hours, 4 hours, 6 hours, hours, 12 hours, and 24 hours post dose. The EDTA blood samples were stored frozen below −18° C. until bioanalysis.

Cyclosporin A blood concentrations were determined by a radioimmunoassay (RIA) method.

The pharmacokinetic parameters Cₘₐₓ (highest observed concentration in blood); tₘₐₓ (time to reach Cₘₐₓ); and AUC(0-24h) (area under the plasma concentration-time curve from 0 to 24 h, calculated by the linear trapezoidal rule, wherein concentrations below the limit of quantitation (LOQ) were taken as ‘zero’), are listed in Table 6.

TABLE 6

| | Composition of | | | | | | |
|---|---|---|---|---|---|---|---|
| | Ex. 1 | Ex. 8 | Ex. 10 | Ex. 11 | Ex. 12 | Ex. 6 | Ex. 13 |
| Actual dose | 4.89 | 4.93 | 4.05 | 4.14 | 4.07 | 3.45 | 4.67 |
| CyA [mg/kg] | | | | | | | |
| AUC (0-24 h) | 1893 | 1973 | 935 | 894 | 576 | 2646 | 3436 |
| [(ng/ml) · h] | | | | | | | |
| Cₘₐₓ [ng/ml] | 394 | 441 | 223 | 195 | 136 | 428 | 635 |
| tₘₐₓ [h] | 1.69 | 1.28 | 1.57 | 1.86 | 2.57 | 1.13 | 1.53 |

### EXAMPLE 17

Single oral doses of 50 mg cyclosporin A per animal of composition of example 6 in form of a suspension in water, corresponding to about 2.5-4 mg/kg were given to fasted dogs (n=10) using a two-block latin square design with a one-week interval between administrations. The nominal doses of cyclosporin A in mg/kg body weight are listed in Table 6.

Blood (about 1 ml each) was collected from the jugular vein at 0 min (=pre-dose), and 10 min, 30 min, 45 min, 1 hour, 1.5 hours, 2 hours, 3 hours, 4 hours, 6 hours, 8 hours, 12 hours, and 24 hours post dose. The EDTA blood samples were stored frozen below −18° C. until bioanalysis.

Cyclosporin A blood concentrations were determined by a radioimmunoassay (RIA) method.

The pharmacokinetic parameters Cₘₐₓ (highest observed concentration in blood); tₘₐₓ (time to reach Cₘₐₓ); and AUC(0-24h) (area under the plasma concentration-time curve from 0 to 24 h, calculated by the linear trapezoidal rule, wherein concentrations below the limit of quantitation (LOQ) were taken as ‘zero’), are listed in Table 6.

### EXAMPLE 18

Single oral doses of 50 mg cyclosporin A per animal of tablet compositions of example 13 corresponding to about 5 mg/kg were given to fasted dogs (n=7) using a two-block latin square design with a one-week interval between administrations. The nominal doses of cyclosporin A in mg/kg body weight are listed in Table 6.

Blood (about 3 ml each) was collected from the cephalic vein at 0 min (=pre-dose), and 10 min, 30 min, 45 min, 1 hour, 1.5 hours, 2 hours, 3 hours, 4 hours, 6 hours, 8 hours, 12 hours, and 24 hours post dose. The EDTA blood samples were stored frozen below −18° C. until bioanalysis.

Cyclosporin A blood concentrations were determined by a radioimmunoassay (RIA) method.

The pharmacokinetic parameters Cₘₐₓ (highest observed concentration in blood); tₘₐₓ (time to reach Cₘₐₓ); and AUC(0-24h) (area under the plasma concentration-time curve from 0 to 24 h, calculated by the linear trapezoidal rule, wherein concentrations below the limit of quantitation (LOQ) were taken as ‘zero’), are listed in Table 6.

1. A solid pharmaceutical composition comprising
(1) a poorly water soluble drug,
(2) a polymer which is solid at room temperature, and
(3) a surfactant which is solid at room temperature and which has a HLB value of between 8 and 17.

2. A composition according to claim 1 wherein the ratio of surfactant: drug is 1:1 to 40.

3. A composition according to claim 1 wherein the surfactant is selected from polyoxyethylene alkyl ethers, polyethoxylated fatty acid esters or polyethylene glycol (PEG) sterol ethers.

4. A composition according to claim 1 wherein the polymer is selected from polyvinyl pyrrolidone; cellulose derivatives such as hydroxypropylmethylcellulose or such as hydroxypropylmethylcellulose phthalate, hydroxypropylmethylcellulose acetate succinate and cellulose acetate phthalate; and poly(meth)acrylates.

5. A solid pharmaceutical composition comprising
(1) a poorly water soluble drug,
(2) a polymer which is solid at room temperature, and
(3) an anionic surfactant which is solid at room temperature.

6. A composition according to claim 5 wherein the anionic surfactant is sodium caprinate or sodium stearoyl lactate.

7. A composition according to claim 5 which is enteric coated.

8. A composition according to claim 1 wherein the composition is in form of a solid dispersion.

9. A composition according to claim 1 wherein the drug is encapsulated in a polymeric matrix.

10. A composition according to claim 1 wherein the poorly water soluble drug is cyclosporin A.

11. A composition according to claim 1 wherein the composition is substantially free of a hydrophilic component.

12. A composition according to claim 1 wherein the composition is substantially free of a lipophilic component.

13. A composition according to claim 1 which upon dilution with an aqueous medium forms a system wherein the poorly water-soluble drug substantially is in the form of fine particles.

14. A composition according to claims 1 which upon dilution with an aqueous medium forms a system which is a mixture of solubilized drug and particulate drug.

15. A composition according to claims 1 which upon dilution with an aqueous medium forms a system wherein the poorly water-soluble drug substantially is solubilized.

16. Use of a composition as claimed in any one of claims 1 the manufacture of a medicament for the treatment of autoimmune diseases or for the use as an immunosuppressant.

17. A process for the production of a composition according to claim 8 which process comprises
(i) dissolving, suspending or dispersing the drug and polymer, if present, in a solvent or solvent mixture,
(ii) adding the surfactant, if present, to the drug/solvent or drug/polymer/solvent mixture,
(iii) evaporating the solvent and co-precipitating the drug with the polymer and/or the surfactant,
(iv) drying the resulting residue, milling and sieving the particles.

18. A process for the production of a composition according to claim 9 which process comprises
(i) preparation of an internal organic phase comprising the drug, the polymer, optionally the surfactant, and an organic solvent,
(ii) preparation of an external aqueous phase comprising a buffered gelatin solution,
(iii) mixing the internal organic phase with the external aqeous phase,
(iv) hardening the microparticles by solvent evaporation.

19. A solid pharmaceutical composition comprising
(1) Cyclosporin A, and
(2) a polymer which is solid at room temperature.

20. A solid pharmaceutical composition comprising
(1) a cyclosporin and
(3) a surfactant which is solid at room temperature.
